(19) United States
(12) Patent Application Publication
(10) Pub. No.: US 2025/0359770 A1
(43) Pub. Date: Nov. 27, 2025

(54) BIOLOGICAL INFORMATION MEASUREMENT DEVICE AND VARIABLE FILTER CIRCUIT

(71) Applicant: Murata Manufacturing Co., Ltd., Nagaokakyo-shi (JP)

(72) Inventors: Naoaki MATSUOKA, Nagaokakyo-shi (JP); Suguru KUSANAGI, Nagaokakyo-shi (JP); Kento FUJIKI, Nagaokakyo-shi (JP); Mana KAWAHARA, Nagaokakyo-shi (JP)

(21) Appl. No.: 19/291,910

(22) Filed: Aug. 6, 2025

Related U.S. Application Data
(63) Continuation of application No. PCT/JP2023/041265, filed on Nov. 16, 2023.

(30) Foreign Application Priority Data
Feb. 10, 2023 (JP) 2023-019369

(51) Int. Cl. A61B 5/024; A61B 5/00; A61B 5/11

(52) U.S. Cl. CPC A61B 5/024; A61B 5/1102; A61B 5/7225; A61B 5/725; A61B 2562/0219

(57) ABSTRACT

A biological information measurement device in which a biological signal with a harmonic structure is input to a variable band-pass filter. A first signal, which has passed through the variable band-pass filter, is input to a frequency calculator. The frequency calculator outputs a second signal including information related to a frequency of the input first signal. A biological information acquirer acquires biological information from the second signal. A band-pass filter controller shifts a passband of the variable band-pass filter based on the information related to the frequency included in the second signal.

### CROSS REFERENCE TO RELATED APPLICATIONS

This application claims the benefit of priority to Japanese Patent Application No. 2023-019369 filed on Feb. 10, 2023 and is a Continuation Application of PCT Application No. PCT/JP2023/041265 filed on Nov. 16, 2023. The entire contents of each application are hereby incorporated herein by reference.

### BACKGROUND OF THE INVENTION

### 1. Field of the Invention

The present invention relates to biological information measurement devices that each analyze biological signals such as pulse waves to measure biological information.

### 2. Description of the Related Art

A biological information measurement device disclosed in International Publication No. WO 2015/045939 includes a phase-locked loop to which biological signals are input. The phase-locked loop includes a phase frequency comparator, a loop filter, and a voltage-controlled oscillator. A signal in a specific frequency band included in a deviation signal that has passed through the loop filter is blocked by a variable low-pass filter. Biological information is acquired from the signal that has passed through the variable low-pass filter.

In addition to signals related to biological information being measured, biological signals also include other signals. For example, in a ballistocardiogram (BCG) obtained to measure a heart rate, signals in the low frequency band caused by respiration or other factors are included. When signals generated by other biological phenomena or environmental factors are superimposed on the signal related to the biological information being measured, these signals become noise, resulting in a decrease in the measurement accuracy of the biological information being measured. Additionally, the frequency of signals related to biological information, such as heart rate, fluctuates over time. To obtain the biological information being measured, it is necessary to analyze signals with frequencies within the expected frequency fluctuation range. It is difficult to remove only the noise with frequencies within the expected frequency fluctuation range without removing the signal related to the biological information being measured. Accordingly, the signal superimposed with a large amount of noise would be analyzed. Due to this noise, the measurement accuracy of the biological information being measured is reduced.

### SUMMARY OF THE INVENTION

Example embodiments of the present invention provide biological information measurement devices each able to reduce or prevent a decrease in the measurement accuracy of biological information even when signals generated by other biological phenomena are superimposed on a signal related to biological information being measured, and variable filter circuits each included in biological information measurement devices.

According to an example embodiment of the present invention a biological information measurement device includes a variable band-pass filter to receive a biological signal having a harmonic structure, a frequency calculator to receive a first signal that has passed through the variable band-pass filter and output a second signal including information related to a frequency of the received first signal, a biological information acquirer to acquire biological information from the second signal, and a band-pass filter controller configured or programmed to shift a passband of the variable band-pass filter based on the information related to the frequency included in the second signal.

According to another example embodiment of the present invention, a variable filter circuit includes a variable band-pass filter with a variable passband a phase-locked loop to generate a tracking signal synchronized with a phase of a signal that has passed through the variable band-pass filter, and a band-pass filter controller configured or programmed to vary a passband of the variable band-pass filter based on a frequency of the tracking signal.

By inputting a signal that has passed through the variable band-pass filter to the frequency calculator among biological signals, it is possible to calculate the frequency without being affected by signals in the frequency band removed by the variable band-pass filter. By varying the passband of the variable band-pass filter based on the frequency of a tracking signal generated by the phase-locked loop, the passband of the variable band-pass filter quickly tracks fluctuations in the frequency of a biological signal. Accordingly, by tracking the frequency fluctuations of a biological signal with large frequency variations, noise removal and frequency calculation are achieved.

The above and other elements, features, steps, characteristics and advantages of the present invention will become more apparent from the following detailed description of the example embodiments with reference to the attached drawings.

### BRIEF DESCRIPTION OF THE DRAWINGS

FIG. 1A includes a block diagram of a biological information measurement device according to a first example embodiment of the present invention, and diagrams each illustrating an example of a signal waveform, and FIG. 1B is a graph illustrating a heartbeat waveform as an example of a biological signal SigB.

FIG. 2A is a block diagram of a variable band-pass filter according to an example embodiment of the present invention, and FIG. 2B is a graph illustrating the bandpass characteristics of the variable band-pass filter.

FIG. 3 is a block diagram of a variable band-pass filter, a frequency calculator, and a band-pass filter controller according to an example embodiment of the present invention.

FIG. 4A is a graph illustrating an example of the spectrum of the biological signal SigB input to the variable band-pass filter, FIGS. 4B and 4C are graphs illustrating examples of the relationship between the spectrum of a signal input to a phase-locked loop of a biological information measurement device according to a comparative example and the passband of a band-pass filter, and FIG. 4D is a graph illustrating an example of the temporal variation of the frequency f₀ of the fundamental wave of the biological signal SigB and the passband Bw of the variable band-pass filter.

FIG. 5A is a graph illustrating an example of the relationship between the passband Bw of a variable band-pass filter of a biological information measurement device according to a first example embodiment and the spectrum of the biological signal SigB, and FIG. 5B is a graph illustrating an example of the temporal variation of the frequency f₀ of the fundamental wave of the biological signal SigB and the passband Bw of the variable band-pass filter.

FIG. 6 is a graph illustrating an example of the temporal variation of the frequency f₀ of the fundamental wave of the biological signal SigB and the passband Bw of a variable band-pass filter according to a reference example.

FIG. 7 is a diagram illustrating an example of the spectrum and signal waveform of the biological signal SigB being measured, as well as a signal SigR superimposed on the biological signal SigB, which is caused by respiration or the like.

FIG. 8 includes a graph illustrating an example of the spectrum obtained by performing spectral analysis on the biological signal SigB, and a block diagram illustrating a function of calculating the frequency f₀ of the fundamental wave from the frequency 2f₀ of the second harmonic wave.

FIG. 9 is a graph illustrating an example of the temporal variation of the fundamental wave (frequency f₀), the second harmonic wave (frequency 2f₀), and the third harmonic wave (frequency 3f₀) of the biological signal SigB, and the passband Bw of the variable band-pass filter.

FIG. 10 is a graph illustrating an example of the temporal variation of the passband Bw of a band-pass filter used in a comparative example, the temporal variation of the frequency 2f₀ of the second harmonic wave of the biological signal SigB, and the temporal variation of the frequency f₀ of the fundamental wave calculated by the biological information acquirer.

FIG. 11 is a block diagram of a biological information measurement device according to a second example embodiment of the present invention.

FIG. 12 is a flowchart illustrating the procedure of processing executed by a signal analyzer.

FIG. 13 is a graph illustrating an example of the spectrum obtained by performing Fourier transform on an input signal.

FIG. 14 is a flowchart illustrating the procedure of control executed by an input controller according to an example embodiment of the present invention.

FIG. 15 includes graphs for explaining control of the input controller.

FIG. 16 is a block diagram of a biological information measurement device according to a third example embodiment of the present invention.

FIG. 17 is a block diagram of a frequency calculator of the biological information measurement device according to the third example embodiment of the present invention.

FIG. 18 is a graph illustrating the temporal variation of a calculation signal Sig2a and a control signal Sig2b included in a second signal Sig2, biological information infB, and the passband Bw of the variable band-pass filter.

### DETAILED DESCRIPTION OF THE EXAMPLE EMBODIMENTS

Example embodiments of the present invention will be described in detail below with reference to the drawings.

### First Example Embodiment

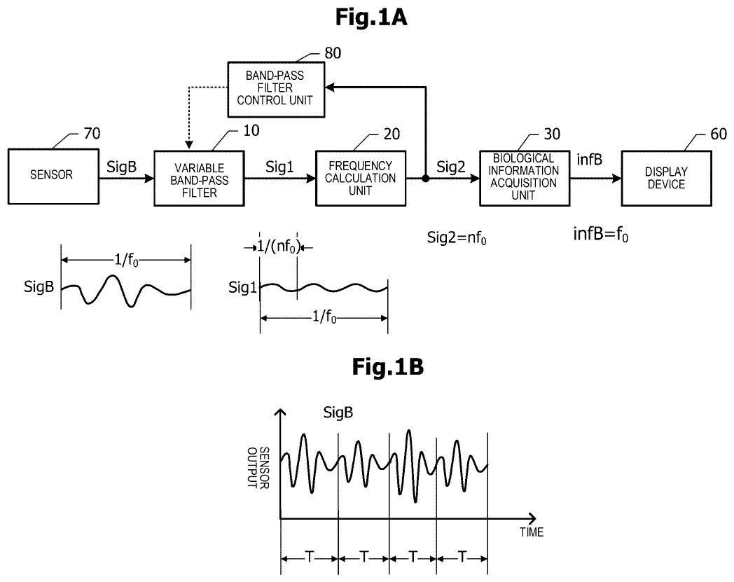

A biological information measurement device according to a first example embodiment of the present invention will be explained with reference to the drawings from FIGS. 1A to 10.

FIG. 1A includes a block diagram illustrating the biological information measurement device according to the first example embodiment, and diagrams each illustrating an example of a signal waveform. FIG. 1B is a graph illustrating a heartbeat waveform as an example of a biological signal SigB. The horizontal axis of FIG. 1B represents time, and the vertical axis represents the sensor output.

The biological information measurement device according to the first example embodiment includes a sensor 70, a variable band-pass filter 10, a frequency calculator 20, a biological information acquirer 30, a display 60, and a band-pass filter controller 80. For example, an acceleration sensor to obtain a ballistocardiogram (BCG) is used as the sensor 70. In addition to the acceleration sensor, other sensors such as, for example, a load sensor, a piezoelectric sensor, etc., may also be used.

In the first example embodiment, it is assumed that the sensor 70 is used either to be placed around the human body, such as on a seat or bed, or to be directly in contact with the human body. Heartbeat vibrations are detected by the sensor 70. The functions of the variable band-pass filter 10, the frequency calculator 20, the biological information acquirer 30, and the band-pass filter controller 80 are provided by software by a micro-controller (MCU). The sensor 70 detects a biological signal, and the biological signal SigB illustrated in FIG. 1B is input to the variable band-pass filter 10. The signal output from the sensor 70 may be an analog signal or a digital signal. In the case where the signal output from the sensor 70 is an analog signal, it is converted to a digital signal by an AD converter within the MCU.

Next, a general formula representing the biological signal SigB will be explained.

For example, when heartbeat signals are captured from a ballistocardiogram, electrocardiogram, pulse wave signals, etc., these signals often mimic a structure including multiple sine waves. It can be confirmed that a signal representing respiration also has the same or similar structure if it is performed periodically. The waveform y′(t) of these periodic biological signals SigB, such as heartbeat or respiration, can often be expressed by the following equation:

$${y}^{′} ( t ) = {\sum }_{r = 0}^{R - 1} {a}_{r} sin ( 2 π {f}_{r} t + {ϕ}_{r} ) \qquad ( 1 )$$

where t represents time, fᵣ represents frequency, φᵣ represents phase, and aᵣ represents amplitude.

When the biological signal SigB includes a fundamental wave with a fundamental frequency f₀ and harmonic waves with frequencies that are kᵣ times the fundamental frequency, the waveform y(t, f₀) of the biological signal SigB can be expressed by the following equation:

$$y ( t , {f}_{0} ) = {a}_{0} sin ( 2 π {f}_{0} t + {ϕ}_{0} ) + {\sum }_{r = 0}^{R - 1} {a}_{r} sin ( 2 π {k}_{r} {f}_{0} t + {ϕ}_{r} ) \qquad ( 2 )$$

The first term on the right side of equation (2) represents the fundamental wave, while the second term represents the harmonic waves.

In biological signals such as heartbeat signals, in addition to intensity variations that depend on time t, the fundamental frequency f₀ also varies. The variation in the fundamental frequency f₀ defines and functions as a factor that alters the heartbeat interval. To represent this alteration in the heartbeat interval, the fundamental frequency f₀ is included as an argument in function y.

In equation (2), when a disturbance is negligible, the amplitude components (a₀, aᵣ), frequency components (f₀, kᵣ), and phase components (φ₀, φᵣ) become characteristic values specific to the vibration transmission path and the biological origin. When the biological signal SigB can be expressed as the sum of the fundamental wave and harmonic waves up to order N, the waveform y(t, f₀) of the biological signal SigB can be expressed by the following equation:

$$y ( t , {f}_{0} ) = {\sum }_{n = 1}^{N} {A}_{n} sin ( 2 π {nf}_{0} t + {ϕ}_{n} ) \qquad ( 3 )$$

For example, the frequency components included in a heartbeat signal (e.g., a BCG waveform) obtained by a specific sensor are, if the maximum order N of its signals is set to 5, f₀, 2f₀, 3f₀, 4f₀, and 5f₀. These frequency components respectively have their own amplitudes A₁, A₂, A₃, A₄, and A₅. At this time, the fundamental frequency f₀ is referred to as the heartbeat frequency, and its reciprocal 1/f₀ is referred to as the heart rate (pulse wave) interval.

In general, the biological signal SigB (FIG. 1B) has a harmonic structure represented by equation (3). That is, the biological signal SigB includes the fundamental wave and its harmonic waves. In FIG. 1B, the period of the fundamental wave of the heartbeat waveform is denoted as T. The period T of the fundamental wave corresponds to the heartbeat interval, and its reciprocal corresponds to the heartbeat frequency.

The variable band-pass filter 10 can shift the passband along the frequency axis under the control of the band-pass filter controller 80. The variable band-pass filter 10 passes a signal within one of the frequency bands, which include the frequency band of the fundamental wave and the frequency bands of multiple harmonic waves, of the input biological signal SigB, while attenuating signals in the other frequency bands. A signal within the frequency band passed by the variable band-pass filter 10 may be referred to as a target signal.

Hereinafter, the case where the passband of the variable band-pass filter 10 corresponds to the frequency band of the n-th harmonic wave of the biological signal SigB will be explained. When n=1, the variable band-pass filter 10 passes a signal within the frequency band of the fundamental wave of the biological signal SigB. In the present specification, the “n-th harmonic wave” when n=1 refers to the fundamental wave. When the fundamental frequency of the biological signal SigB is f₀, the waveform of a first signal Sig1, which has passed through the variable band-pass filter 10, will have a shape close to a sine wave with a frequency of nf₀.

The frequency calculator 20 outputs a second signal Sig2 including information related to the frequency nf₀ of the first signal Sig1, which has passed through the variable band-pass filter 10. For example, the second signal Sig2 has the value of the frequency nf₀. When the frequency calculator 20 includes an analog circuit, the second signal Sig2 has a voltage value corresponding to the frequency nf₀.

The biological information acquirer 30 acquires biological information infB from the second signal Sig2. For example, the biological information acquirer 30 calculates the fundamental frequency f₀ from the second signal Sig2. When the biological signal SigB is a heartbeat signal, the biological information infB is the heartbeat frequency, and its value is provided by the fundamental frequency f₀. Additionally, the heartbeat interval is provided by its reciprocal 1/f₀.

The display 60 displays information related to the biological information infB acquired by the biological information acquirer 30. For example, the display 60 displays the heartbeat frequency or heartbeat interval as a numerical value or in a graph.

The band-pass filter controller 80 varies the passband of the variable band-pass filter 10 based on the frequency nf₀ represented by the second signal Sig2 calculated by the frequency calculator 20. For example, the band-pass filter controller 80 shifts the passband along the frequency axis such that the center frequency of the passband becomes equal or substantially equal to nf₀, which is the value of the second signal Sig2. Here, the term “shift” includes both cases: one where the center frequency is shifted without changing the bandwidth, and another where the bandwidth changes additionally along with the shift in the center frequency.

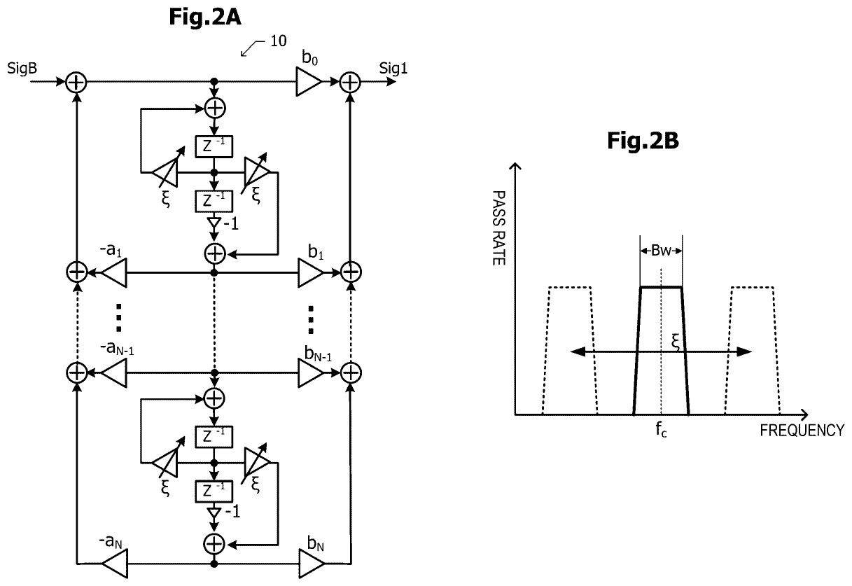

FIG. 2A is a block diagram of the variable band-pass filter 10, and FIG. 2B is a graph illustrating the bandpass characteristics of the variable band-pass filter 10. The variable band-pass filter 10 is designed such that a filter designed as a low-pass filter can be configured as a variable band-pass filter. The method of using a filter designed as a low-pass filter as a band-pass filter is referred to as low-pass to band-pass transformation (LP-BP). As the variable band-pass filter 10, a digital filter of the infinite impulse response (IIR) type is used. The center frequency of the passband is denoted as f_{c}, and the passband is denoted as Bw.

When the center frequency f_{c} is greater than or equal to 0 Hz and less than or equal to the bandwidth of the passband Bw, the bandpass characteristics have the shape of a low-pass filter. When the center frequency f_{c} is higher than the bandwidth of the passband Bw, the bandpass characteristics have the shape of a nearly bilaterally symmetric band-pass filter. However, the shape of the transmission characteristics may differ slightly depending on the number of taps in the filter and the type of filter shape (e.g., Chebyshev filter type). It is not necessary for the variable band-pass filter 10 used in the present example embodiment to have an LP-BP configuration. Additionally, the passband Bw can be changed not only by adjusting the center frequency f_{c}, but also by modifying the cutoff frequencies on both the upper and lower sides of the passband Bw. Alternatively, the passband Bw can be changed by modifying both of the center frequency f_{c} and the cutoff frequencies of the passband Bw.

The input to the variable band-pass filter 10 is a digital value obtained by sampling the biological signal SigB at a predetermined sampling frequency, and the output is the digital value of the first signal Sig1 filtered by the variable band-pass filter 10. In FIG. 2A, Z⁻¹ is a delay block, while a₁, a₂, . . . a_{N}, and b₀, b₁, . . . and b_{N} are filter parameters that determine the filter shape. The values of these filter parameters can be determined as those of an IIR-type low-pass filter with the passband Bw.

A coefficient ξ is a coefficient used to change the center frequency f_{c} of the passband. As illustrated in FIG. 2B, when the coefficient ξ is changed, the center frequency f_{c} shifts along the frequency axis.

The filter shape of the variable band-pass filter 10 is fixed. For example, it is preferable to perform spectral analysis on the expected biological signal SigB and determine the filter shape based on the spectral shape. Based on the determined filter shape, it is preferable to determine the values of the filter parameters a₁, a₂, . . . a_{N}, and b₀, b₁, . . . and b_{N}.

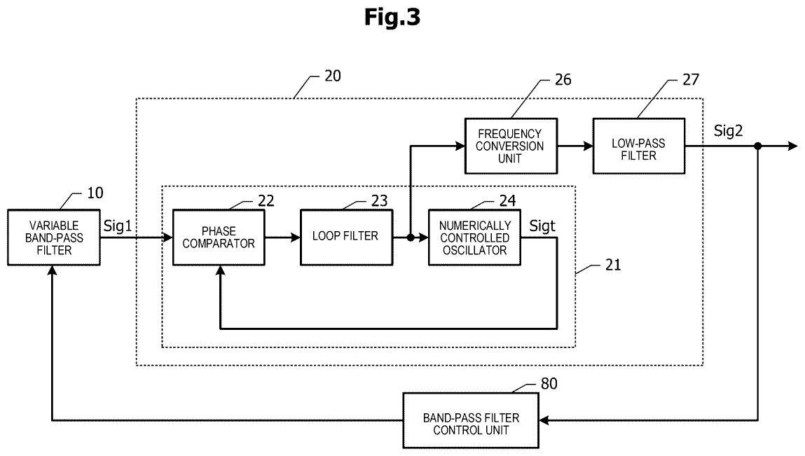

The functions of the frequency calculator 20 will now be explained with reference to FIG. 3. FIG. 3 is a block diagram of the variable band-pass filter 10, the frequency calculator 20, and the band-pass filter controller 80. The functions of the individual blocks of the frequency calculator 20 are provided, for example, with software. These functions can also be provided with hardware circuits.

The variable band-pass filter 10 passes a signal in a predetermined specific-order frequency band among the frequency bands of the fundamental wave and multiple harmonic waves of the biological signal SigB. The first signal Sig1, which has passed through the variable band-pass filter 10, is input to the frequency calculator 20.

The frequency calculator 20 includes a phase-locked loop 21, a frequency converter 26, and a low-pass filter 27. The phase-locked loop 21 includes a phase comparator 22, a loop filter 23, and a numerically controlled oscillator 24. The phase-locked loop 21 is designed to be able to track a signal in the frequency band passed by the variable band-pass filter 10, among the frequency bands of the fundamental wave and multiple harmonic waves of the biological signal SigB (FIGS. 1A and 1B).

The numerically controlled oscillator 24 varies the frequency and phase of the tracking signal Sigt to be output, in accordance with the output of the loop filter 23. The range of the initial frequency at the start of operation of the numerically controlled oscillator 24 and the frequency that the tracking signal Sigt tracks (hereinafter sometimes referred to as the tracking frequency) can be initialized by an external control signal. Additionally, the operation of the phase-locked loop 21 can be stopped (tracking can be halted) by an external control signal. As the phase-locked loop 21, a free-run phase-locked loop that can track a specific frequency band without parameter settings or input of an external control signal may be used. Furthermore, for example, when the function of the phase-locked loop 21 is provided with a hardware circuit, a voltage-controlled oscillator is used instead of the numerically controlled oscillator 24.

The phase comparator 22 compares the input first signal Sig1 with the tracking signal Sigt output from the numerically controlled oscillator 24 and calculates the phase difference. The loop filter 23 outputs an appropriate control signal to control the numerically controlled oscillator 24 based on the phase difference calculated by the phase comparator 22.

The frequency converter 26 converts the control value of the control signal output from the loop filter 23 to frequency information. More specifically, the control value of the control signal input to the numerically controlled oscillator 24 is converted to a tracking frequency of the current phase-locked loop 21. Depending on the configuration of the loop filter 23 and the numerically controlled oscillator 24, the output of the loop filter 23 may include frequency information. In such a case, the frequency converter 26 is unnecessary.

The low-pass filter 27 smooths the temporal variation of the control value of the control signal output from the loop filter 23. For example, depending on the design of the loop filter 23 and the numerically controlled oscillator 24, ripple noise of a non-negligible magnitude may be superimposed on the output of the loop filter 23. The low-pass filter 27 is installed to remove this ripple noise. If, depending on the design of the loop filter 23 and the numerically controlled oscillator 24, it is possible to reduce or prevent the ripple noise to a level where it can be ignored, or the ripple noise is not a problem in subsequent display control or applications, the low-pass filter 27 may be omitted.

In the first example embodiment, as an example, the initial frequency of the phase-locked loop 21 is set to about 2.5 Hz, and the range of the tracking frequency of the numerically controlled oscillator 24 is set to be greater than or equal to about 2 Hz and less than or equal to about 4 Hz, for example. A fourth-order IIR digital filter is used as the low-pass filter 27, and the cutoff frequency of the low-pass filter 27 is set to about 0.6 Hz, for example. If a large output delay is not a problem or steep cutoff characteristics are not required, for example, an FIR digital filter may be used as the low-pass filter 27.

The second signal Sig2, that is, the value of the tracking frequency, is input to the band-pass filter controller 80. The band-pass filter controller 80 controls the variable band-pass filter 10 so that the center frequency f_{c} of the passband of the variable band-pass filter 10 becomes equal or substantially equal to the tracking frequency. Specifically, the coefficient ξ (FIG. 2A) of the variable band-pass filter 10 where the center frequency f_{c} (FIG. 2B) becomes equal or substantially equal to the tracking frequency is determined, and the new value of the coefficient ξ is set in the variable band-pass filter 10.

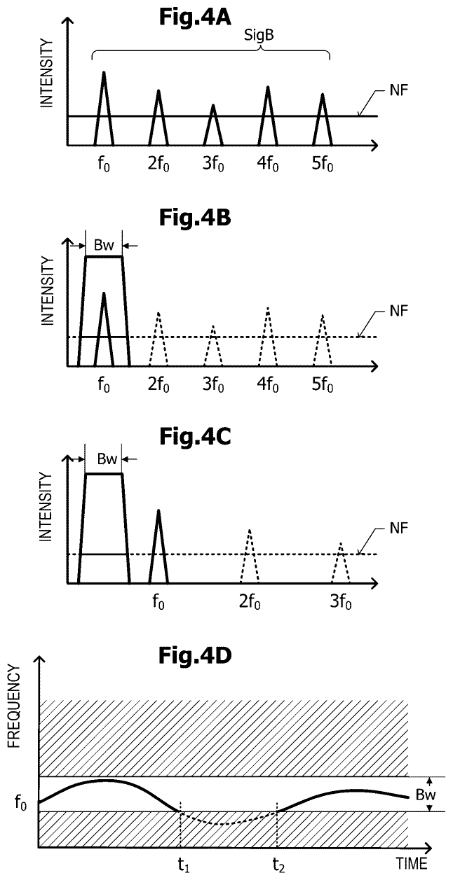

Next, the excellent effects of the first example embodiment, compared with a comparative example, will be explained with reference to the drawings from FIG. 4A to FIG. 4D.

FIG. 4A is a graph illustrating an example of the spectrum of the biological signal SigB input to the variable band-pass filter 10 (FIG. 1A). FIG. 4A illustrates an example where the biological signal SigB is a BCG heartbeat signal. The horizontal axis represents frequency, and the vertical axis represents intensity. The biological signal SigB includes the fundamental wave at frequency f₀, the second harmonic wave at frequency 2f₀, the third harmonic wave at frequency 3f₀, the fourth harmonic wave at frequency 4f₀, and the fifth harmonic wave at frequency 5f₀.

The noise floor NF, caused by environmental noise such as thermal noise, is superimposed on the spectrum of the fundamental wave and harmonic waves. Furthermore, when acquiring heartbeat signals using a sensor such as an acceleration sensor, a load sensor, or the like, for example, large noise that reduces the signal-to-noise ratio (SNR) to below 0 dB may be superimposed due to the effects of body movement or external vibrations, resulting in a large noise floor NF superimposed on the frequency band of the heartbeat signal. As described above, if non-negligible noise is superimposed on the frequency band of the heartbeat signal, a large error may occur in the tracking frequency of the phase-locked loop 21 (FIG. 3).

FIGS. 4B and 4C are graphs illustrating examples of the relationship between the spectrum of a signal input to the phase-locked loop 21 of a biological information measurement device according to a comparative example, which can reduce this error, and the passband of a band-pass filter. In this comparative example, instead of the variable band-pass filter 10 (FIGS. 1A and 3), a band-pass filter with a fixed passband is used. The passband Bw of this band-pass filter, as illustrated in FIG. 4B, includes the frequency band of the fundamental wave at frequency f₀, and does not include the frequency bands of the second and higher-order harmonic waves. Therefore, only the fundamental wave is input to the phase-locked loop 21 (FIG. 3).

Noise within the passband Bw is input to the phase-locked loop 21, but noise and harmonic waves outside the range of the passband Bw are not input to the phase-locked loop 21. This can reduce the effect of noise and improve the calculation accuracy of the frequency f₀ of the fundamental wave.

When the heartbeat frequency of the biological signal SigB being measured fluctuates, as illustrated in FIG. 4C, the frequency f₀ the fundamental wave varies. This may cause the frequency band of the fundamental wave to fall outside the passband Bw of the band-pass filter.

FIG. 4D is a graph illustrating an example of the temporal variation of the frequency f₀ of the fundamental wave of the biological signal SigB and the passband Bw of the band-pass filter used in the comparative example. The horizontal axis represents time, and the vertical axis represents frequency. In FIG. 4D, regions outside the range of the passband Bw are marked with hatching. For example, during the period from time t₁ to t₂, the frequency f₀ is outside the passband Bw.

When the frequency f₀ of the fundamental wave falls outside the passband Bw of the band-pass filter, the fundamental wave of the biological signal SigB is no longer input to the phase-locked loop 21 (FIG. 3), thus disabling the measurement of the frequency of the fundamental wave of the biological signal SigB.

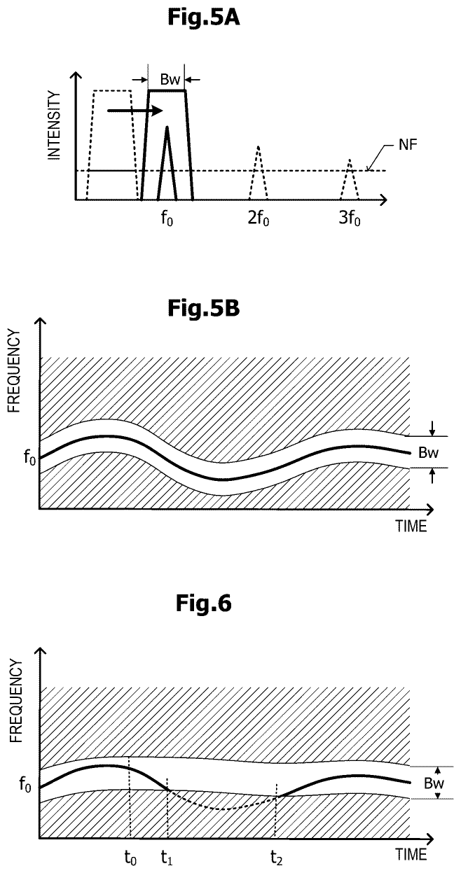

FIG. 5A is a graph illustrating an example of the relationship between the passband Bw of the variable band-pass filter 10 of the biological information measurement device according to the first example embodiment and the spectrum of the biological signal SigB. The horizontal axis represents frequency, and the vertical axis represents intensity. In the first example embodiment, when the frequency f₀ of the fundamental wave of the biological signal SigB varies, the passband Bw of the variable band-pass filter 10 shifts in response to the variation in frequency f₀.

FIG. 5B is a graph illustrating an example of the temporal variation of the frequency f₀ of the fundamental wave of the biological signal SigB and the passband Bw of the variable band-pass filter 10. The horizontal axis represents time, and the vertical axis represents frequency. In FIG. 5B, regions outside the range of the passband Bw are marked with hatching. The passband Bw fluctuates in response to the fluctuation of the frequency f₀ of the fundamental wave of the biological signal SigB. Therefore, even if the frequency f₀ of the fundamental wave fluctuates, the fundamental wave of the biological signal SigB is always input to the phase-locked loop 21 (FIG. 3).

As described above, in the first example embodiment, only the fundamental wave of the biological signal SigB and the noise within the passband Bw are input to the phase-locked loop 21 (FIG. 3), while the second and higher-order harmonic waves and unwanted noise outside the passband Bw are not input to the phase-locked loop 21. Therefore, the phase-locked loop 21 becomes less susceptible to the effects of harmonic waves other than the fundamental wave, which is the target to be tracked, as well as noise, enabling the highly accurate calculation of the frequency f₀ of the fundamental wave.

Furthermore, even if the frequency f₀ of the fundamental wave of the biological signal SigB fluctuates, the passband Bw of the variable band-pass filter 10 tracks the fluctuation of the frequency f₀ of the fundamental wave, thus enabling the accurate calculation of the frequency f₀ of the fundamental wave at all times.

Although FIG. 5A illustrates an example where the variable band-pass filter 10 passes the fundamental wave of the biological signal SigB, as explained with reference to FIG. 1A, the variable band-pass filter 10 may pass the n-th harmonic wave of the biological signal SigB. In this case, the frequency indicated by the second signal Sig2 output from the frequency calculator 20 (FIG. 3) is nf₀. The band-pass filter controller 80 controls the variable band-pass filter 10 so that the center frequency of the variable band-pass filter 10 tracks the frequency nf₀ of the n-th harmonic wave and varies accordingly.

Next, the excellent effects of the first example embodiment, compared with a reference example, will be explained with reference to FIG. 6. In this reference example, the variable band-pass filter 10 is used, as in the first example embodiment. The variable band-pass filter 10 according to the reference example uses an adaptive operation that varies the center frequency of the variable band-pass filter 10 according to the frequency of the input signal using a least mean squares (LMS) algorithm. The variable band-pass filter explained in the first example embodiment is described in “Shunsuke KOSHITA at el. 2013, Adaptive IIR Band-Pass/Band-Stop Filtering Using High-Order Transfer Function and Frequency Transformation, Interdisciplinary Information Sciences Vol. 19, No. 2 (2013) 163-172”, which also explains the variable band-pass filter's tracking operation with the input signal using the LMS algorithm.

In the method of applying the LMS algorithm to change the center frequency of the variable band-pass filter 10, the convergence of the passband of the variable band-pass filter 10 is slow, and there are cases where the center frequency of the passband Bw cannot track variations in the frequency of the input signal. Depending on the parameter settings of the variable band-pass filter 10, it is possible to adjust the tracking ability and convergence. However, if the noise in the input signal becomes large, it becomes difficult to have the center frequency of the passband track the frequency of the input signal.

FIG. 6 is a graph illustrating an example of the temporal variation of the frequency f₀ of the fundamental wave of the biological signal SigB and the passband Bw of the variable band-pass filter 10 according to the reference example. The horizontal axis represents time, and the vertical axis represents frequency. In FIG. 6, regions outside the range of the passband Bw are marked with hatching. In the example illustrated in FIG. 6, from time t₀, the center frequency of the passband Bw is unable to track the frequency of the input signal. As a result, during the interval from time t₁ to t₂, the frequency of the input signal falls outside the passband Bw.

In contrast, in the first example embodiment, the variable band-pass filter 10 is controlled based on the frequency of a tracking signal generated by the phase-locked loop 21 (FIG. 3). Therefore, it is possible to have the center frequency of the passband Bw sufficiently track the fluctuation of the frequency f₀ of the fundamental wave of the biological signal SigB.

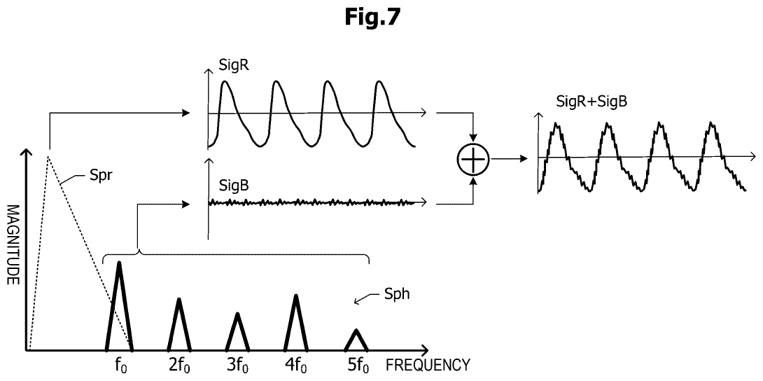

Referring now to the drawings from FIGS. 7 to 10, the excellent effects of the case in which the variable band-pass filter 10 passes the second harmonic wave of the biological signal SigB will be explained.

FIG. 7 is a diagram illustrating an example of the spectrum and signal waveform of the biological signal SigB being measured, as well as a signal SigR superimposed on the biological signal SigB, which is caused by respiration or the like.

Each of the peaks of the fundamental wave at frequency f₀, the second harmonic wave at frequency 2f₀, the third harmonic wave at frequency 3f₀, the fourth harmonic wave at frequency 4f₀, and the fifth harmonic wave at frequency 5f₀ appears in the spectrum Sph of the biological signal SigB. Furthermore, the spectrum Spr of the signal SigR, caused by respiration or the like, is present. A portion of the frequency band of the spectrum Spr overlaps with the frequency band of the fundamental wave of the biological signal SigB.

For example, the heart rate is generally said to be greater than or equal to about 60 bpm and less than or equal to about 85 bpm, which, when converted to frequency, means that the heartbeat frequency is greater than or equal to about 1 Hz and less than or equal to about 1.4 Hz. On the other hand, for example, the respiration rate is generally greater than or equal to about 12 bpm and less than or equal to about 20 bpm, which, when converted to frequency, means that the respiration frequency is greater than or equal to about 0.2 Hz and less than or equal to about 0.3 Hz. In terms of frequency, the two do not overlap. However, on the surface of the body, the movement caused by respiration is often several times larger than the movement caused by the heartbeat. When movements on the body surface are captured with an acceleration sensor, the signal caused by movements due to respiration tends to appear relatively larger.

Additionally, the signal SigR caused by respiration takes the shape of a gradual triangular wave, and as illustrated in FIG. 7, its harmonic waves can extend into the frequency band of the fundamental wave of the biological signal SigB, while maintaining a large amplitude level. When a signal synthesized from the signal SigR caused by respiration or the like and the biological signal SigB caused by the heartbeat is directly input to the phase-locked loop 21 (FIG. 3), it becomes difficult for the frequency of the tracking signal Sigt (FIG. 3) to accurately track the frequency of the fundamental wave of the biological signal SigB caused by the heartbeat, due to the effect of the signal SigR caused by respiration or the like.

When the variable band-pass filter 10 is set to input, instead of the fundamental wave of the biological signal SigB, the second harmonic wave to the frequency calculator 20 (FIG. 1A), the fundamental wave of the biological signal SigB and the signal SigR caused by respiration or the like are attenuated by the variable band-pass filter 10 (FIG. 1A), and they are not input to the frequency calculator 20 (FIG. 1A). Therefore, the frequency 2f₀ of the second harmonic wave can be calculated with high accuracy without being adversely affected by the signal SigR caused by respiration or the like. Since the biological information acquirer 30 (FIG. 1A) obtains the frequency f₀ of the fundamental wave from the frequency 2f₀ calculated with high accuracy by the frequency calculator 20, the frequency f₀ of the fundamental wave can be obtained with high accuracy without being adversely affected by the signal SigR caused by respiration or the like.

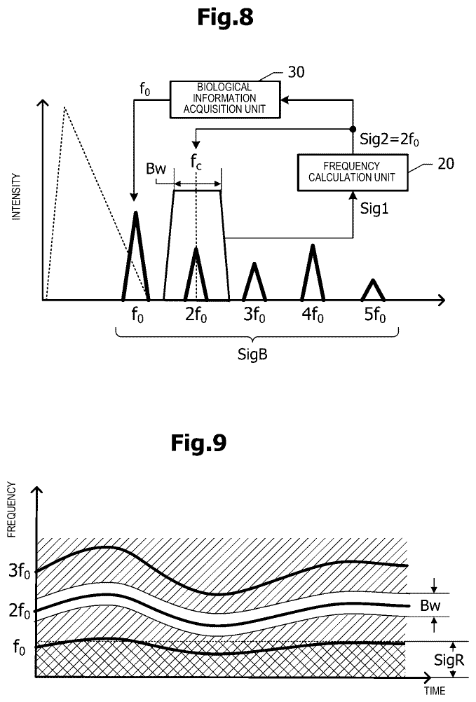

FIG. 8 includes a graph illustrating an example of the spectrum obtained by performing spectral analysis on the biological signal SigB, and a block diagram illustrating calculation of the frequency f₀ of the fundamental wave from the frequency 2f₀ of the second harmonic wave. The horizontal axis of the graph represents frequency, and the vertical axis represents intensity.

The biological signal SigB includes the fundamental wave at frequency f₀, and the second to fifth harmonic waves at 2f₀, 3f₀, 4f₀, and 5f₀. The variable band-pass filter 10 (FIG. 1) passes a signal in the passband Bw including the frequency 2f₀ of the second harmonic wave. The lower cutoff frequency on the low frequency side of the variable band-pass filter 10 is higher than the fundamental frequency f₀, and the higher cutoff frequency on the high frequency side is lower than the frequency 3f₀ of the third harmonic wave. That is, the variable band-pass filter 10 passes only the frequency band of the second harmonic wave among the frequency bands of the fundamental wave and multiple harmonic waves of the biological signal SigB, while attenuating the fundamental wave and the third and higher harmonic waves. Furthermore, the variable band-pass filter 10 attenuates the signal SigR caused by respiration or the like.

Using the first signal Sig1 (second harmonic wave) having passed through the variable band-pass filter 10, the frequency calculator 20 obtains its frequency 2f₀, and outputs the value of the frequency 2f₀ as the second signal Sig2. The order of the harmonic wave that the frequency calculator 20 tracks and calculates is referred to as the target order n. In the example illustrated in FIG. 8, the target order n is 2, for example. The biological information acquirer 30 calculates the fundamental frequency f₀ by dividing the value indicated by the second signal Sig2 by the target order n, i.e., 2.

Based on the second signal Sig2 output from the frequency calculator 20, the center frequency f_{c} of the passband Bw of the variable band-pass filter 10 (FIG. 1) is controlled. For example, the center frequency f_{c} of the passband Bw tracks variations in the frequency 2f₀ calculated by the frequency calculator 20 and varies accordingly.

Although the order n of the harmonic wave passed by the variable band-pass filter 10 is 2 in FIG. 8, in the case where n=3, 4, or 5, for example, the fundamental frequency f₀ can be obtained through similar processing. As described above, the fundamental frequency f₀ is calculated without using the signal in the frequency band of the fundamental wave of the biological signal SigB. In the case where the variable band-pass filter 10 passes the signal in the frequency band of the fundamental wave and attenuates the signals in the frequency bands of the second and higher harmonic waves, the value of the second signal Sig2 output from the frequency calculator 20 will be equal or substantially equal to the fundamental frequency f₀.

FIG. 9 is a graph illustrating an example of the temporal variation of the fundamental wave (frequency f₀), the second harmonic wave (frequency 2f₀), and the third harmonic wave (frequency 3f₀) of the biological signal SigB, and the passband Bw of the variable band-pass filter 10. The horizontal axis represents time, and the vertical axis represents frequency. In FIG. 9, regions outside the range of the passband Bw of the variable band-pass filter 10 are marked with right-slanting hatching. The frequency band of the signal SigR caused by respiration, illustrated in FIG. 7, is marked with left-slanting, relatively light hatching.

Since the center frequency of the passband Bw of the variable band-pass filter 10 tracks the frequency 2f₀ of the second harmonic wave and varies accordingly, the frequency band of the second harmonic wave is included in the range of the passband Bw at all times. The frequency bands of the fundamental wave and third and higher harmonic waves of the biological signal SigB are outside the range of the passband Bw. Additionally, the frequency band of the signal SigR caused by respiration, illustrated in FIG. 7, is also outside the passband Bw.

Therefore, the frequency calculator 20 (FIG. 8) can highly accurately obtain the frequency 2f₀ of the second harmonic wave even when the frequency f₀ of the fundamental wave fluctuates over time. Since the biological information acquirer 30 calculates the frequency f₀ by dividing the value of the frequency 2f₀, which has been obtained with high accuracy, by the target order n=2, for example, the frequency f₀ of the fundamental wave of the biological signal SigB can be obtained with high accuracy. The target order n may be set to 3 or greater.

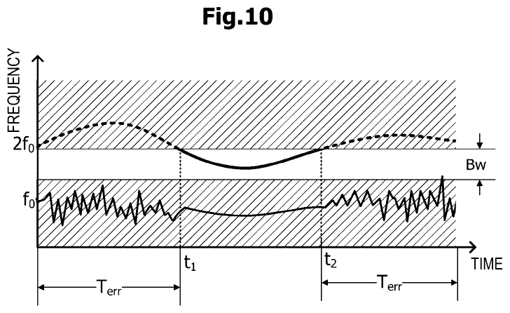

Next, the excellent effects of the first example embodiment in the case where the target order n is set to 2, compared with a comparative example using, instead of the variable band-pass filter 10 (FIGS. 1A and 3), a band-pass filter with a fixed center frequency of the passband, will be explained with reference to FIG. 10.

FIG. 10 is a graph illustrating an example of the temporal variation of the passband Bw of a band-pass filter used in a comparative example, the temporal variation of the frequency 2f₀ of the second harmonic wave of the biological signal SigB, and the temporal variation of the frequency f₀ of the fundamental wave calculated by the biological information acquirer 30. The horizontal axis represents time, and the vertical axis represents frequency.

Although the frequency 2f₀ of the second harmonic wave of the biological signal SigB fluctuates over time, the center frequency of the passband Bw of the band-pass filter is fixed. Therefore, the frequency band of the second harmonic wave falls within the range of the passband Bw in a partial interval (e.g., from time t₁ to t₂), but it is outside the range of the passband Bw in other intervals Tₑᵣᵣ.

In the intervals Tₑᵣᵣ, the frequency calculator 20 (FIG. 8) is unable to highly accurately calculate the frequency 2f₀ of the second harmonic wave. As a result, the accuracy of the frequency f₀ of the fundamental wave obtained by the biological information acquirer 30 (FIG. 8) decreases. Therefore, as illustrated in FIG. 10, during the intervals Tₑᵣᵣ, a large noise is superimposed on the frequency f₀ of the fundamental wave acquired by the biological information acquirer 30.

In contrast, in the first example embodiment, as illustrated in FIG. 9, the frequency 2f₀ of the second harmonic wave remains within the range of the passband Bw of the variable band-pass filter 10 at all times. Therefore, a decrease in the accuracy of the frequency f₀ of the fundamental wave acquired by the biological information acquirer 30 (FIG. 8) can be reduced or prevented.

Next, a modification of the first example embodiment will be explained.

In the first example embodiment, the phase-locked loop 21 configured to make the frequency of the tracking signal Sigt equal or substantially equal to the frequency of the first signal Sig1 is used. Alternatively, for example, a frequency multiplier phase-locked loop may be used. In the case of using a frequency multiplier phase-locked loop as the phase-locked loop 21, a frequency divider is provided between the output of the numerically controlled oscillator 24 and the input of the phase comparator 22, and the tracking signal Sigt is divided by a factor of m using the frequency divider. The numerically controlled oscillator 24 generates the tracking signal Sigt with a frequency m times the frequency of the first signal Sig1.

### Second Example Embodiment

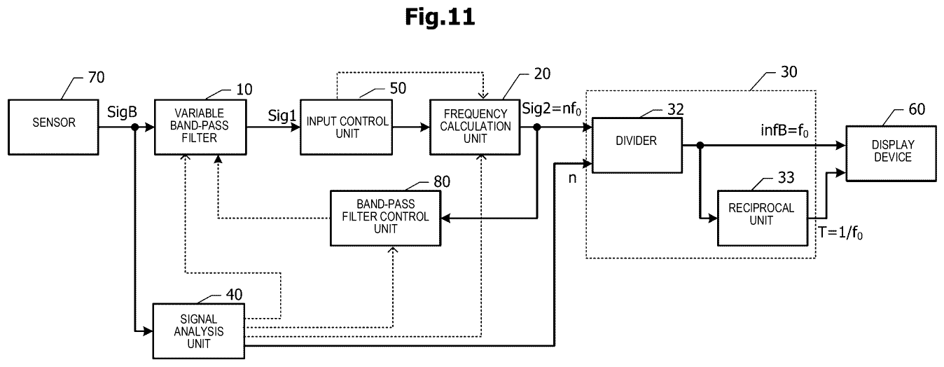

Next, a biological information measurement device according to a second example embodiment of the present invention will be explained with reference to the drawings from FIGS. 11 to 15. Hereinafter, the explanation for the components that are common to the biological information measurement device according to the first example embodiment, as explained with reference to the drawings from FIGS. 1A to 10, will be omitted.

FIG. 11 is a block diagram of the biological information measurement device according to the second example embodiment. The biological information measurement device according to the second example embodiment includes, in addition to the sensor 70, the variable band-pass filter 10, the frequency calculator 20, the biological information acquirer 30, the display 60, and the band-pass filter controller 80 of the biological information measurement device according to the first example embodiment, a signal analyzer 40 and an input controller 50. The biological signal SigB is input to the signal analyzer 40.

In the first example embodiment, the order n of the harmonic wave (target order n) passed by the variable band-pass filter 10 is predetermined. In the second example embodiment, the signal analyzer 40 analyzes the biological signal SigB to determine the target order n.

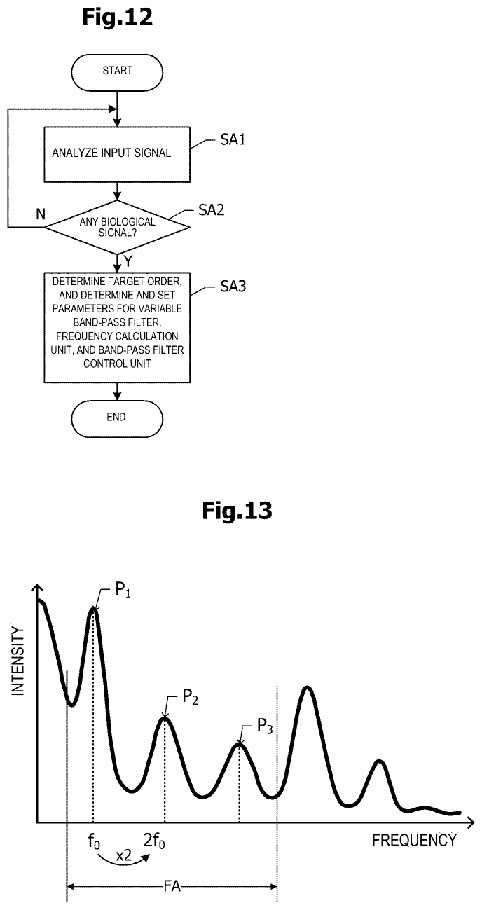

Next, the procedure of processing executed by the signal analyzer 40 will be explained with reference to FIG. 12. FIG. 12 is a flowchart illustrating the procedure of processing executed by the signal analyzer 40.

First, when the human body is at rest, the signal analyzer 40 analyzes the input signal (step SA1). Based on the analysis result, it is determined whether the biological signal SigB such as a heartbeat signal is included in the input signal (step SA2). For example, in step SA1, the input signal is Fourier transformed, and if the result of the Fourier transform has a harmonic structure represented by equation (3), it is determined in step SA2 that the biological signal SigB is present. Alternatively, in step SA1, the root mean square (RMS) of the intensity of the input signal is calculated, and if the calculated result is greater than or equal to a threshold, it is determined in step SA2 that the biological signal SigB is present.

If the biological signal SigB is not included in the input signal, step SA1 and step SA2 are repeated until it is determined that the biological signal SigB is present. If it is determined that the biological signal SigB is included in the input signal, the target order n is determined, and various parameters for the variable band-pass filter 10, the frequency calculator 20, and the band-pass filter controller 80 are determined and set (step SA3). The target order n is information used as the basis for the frequency calculator 20 to output the second signal Sig2, which includes information related to the frequency of the first signal Sig1. Furthermore, the target order n is information used as the basis for the biological information acquirer 30 to acquire biological information infB from the second signal Sig2. As described above, “information used as the basis” refers to, for example, parameters used to obtain output information from input information.

For example, the order of the peak appearing in the frequency band with the smallest noise floor in the spectrum of the biological signal SigB (FIG. 4A) may be determined as the target order n. For example, the cutoff frequency of the variable band-pass filter 10, the initial frequency of the numerically controlled oscillator 24 (FIG. 3), and the parameter for the loop filter 23 are determined and set.

Next, an example of a method of determining the initial value of the center frequency of the passband of the variable band-pass filter 10 will be explained with reference to FIG. 13. FIG. 13 is a graph illustrating an example of the spectrum obtained by performing Fourier transform on the input signal. The horizontal axis represents frequency, and the vertical axis represents intensity. An analysis target frequency range FA is predetermined. The signal analyzer 40 detects the peak of the spectrum within the analysis target frequency range FW. In the example illustrated in FIG. 3, peaks P₁, P₂, and P₃ are detected. Among the detected peaks P₁, P₂, and P₃, the peak P₁ on the lowest frequency side is used as the peak of the fundamental wave.

A value obtained by multiplying the frequency f₀ of the fundamental wave by the target order n is adopted as the initial value of the center frequency. When the target order n is 2, the frequency 2f₀, which is twice the frequency f₀ of the fundamental wave, is used as the initial value of the center frequency of the passband of the variable band-pass filter 10 and as the initial value for the phase-locked loop 21 (FIG. 3).

The reciprocal of the average peak-to-peak interval of the waveform of the biological signal SigB when the biological signal SigB is stable may be used as the frequency of the fundamental wave.

When these parameters are set to operate the biological information measurement device, the value of the second signal Sig2 output from the frequency calculator 20 (FIG. 6) becomes the frequency nf₀ of the n-th harmonic wave.

In the second example embodiment, the biological information acquirer 30 (FIG. 11) includes a divider 32 and a reciprocator 33. The signal analyzer 40 notifies the divider 32 of the target order n. The divider 32 divides the frequency nf₀, indicated by the second signal Sig2 input from the frequency calculator 20, by the target order n to generate biological information infB indicating the value of the frequency f₀ of the fundamental wave of the biological signal SigB. The biological information infB represents the heartbeat frequency. The reciprocator 33 calculates the reciprocal of the biological information infB, output from the divider 32, to generate information T representing the heartbeat interval. The obtained heartbeat frequency and heartbeat interval information is displayed on the display 60.

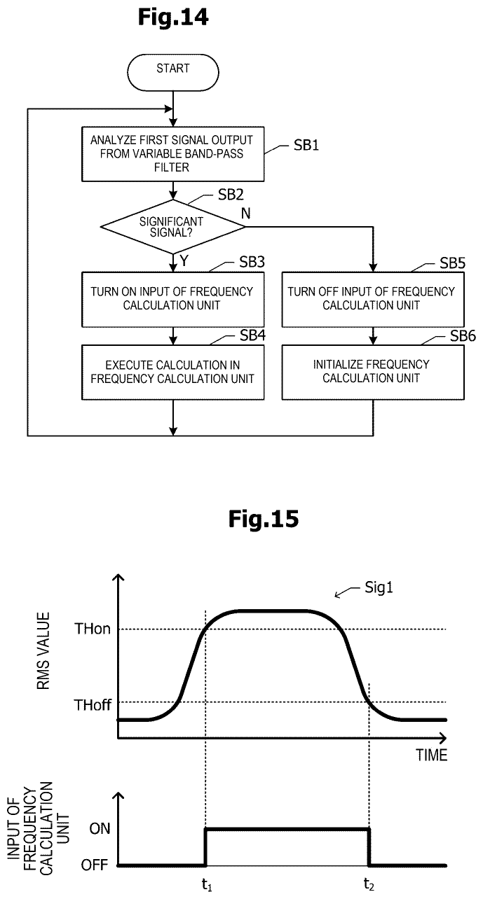

Next, the function of the input controller 50 (FIG. 11) will be explained with reference to FIG. 14. FIG. 14 is a flowchart illustrating the procedure of control executed by the input controller 50.

The input controller 50 (FIG. 11) analyzes the first signal Sig1 output from the variable band-pass filter 10 (step SB1). Based on the analysis result, it is determined whether the variable band-pass filter 10 is outputting a first signal Sig1 of a magnitude that can be subjected to calculation by the frequency calculator 20 (hereinafter referred to as the significant first signal Sig1) (step SB2). If the variable band-pass filter 10 is outputting the significant first signal Sig1, the signal input to the frequency calculator 20 is turned on (step SB3). Accordingly, the frequency calculator 20 performs an operation to calculate the frequency (step SB4). If the variable band-pass filter 10 is not outputting the significant first signal Sig1, the signal input to the frequency calculator 20 is turned off (step SB5). That is, no signal is input to the frequency calculator 20. Furthermore, the frequency calculator 20 is initialized (step SB6).

Next, control performed by the input controller 50 (FIG. 11) will be explained with reference to FIG. 15. FIG. 15 includes graphs for explaining control of the input controller 50. The upper graph in FIG. 15 represents the temporal variation of the root mean square (RMS) of the intensity of the first signal Sig1 output from the variable band-pass filter 10, while the lower graph represents the timing chart for turning the signal input to the frequency calculator 20 (FIG. 11) on and off. The horizontal axis represents time, while the vertical axis of the upper graph represents RMS, and the vertical axis of the lower graph represents turning the signal input to the frequency calculator 20 on and off.

The input controller 50 (FIG. 11) calculates the RMS of the first signal Sig1. When the signal input to the frequency calculator 20 is turned off at the current time, if the RMS value becomes greater than or equal to an on-threshold THon (time t₁), the signal input to the frequency calculator 20 is switched from off to on. When the signal input to the frequency calculator 20 is turned on at the current time, if the RMS value becomes less than or equal to an off-threshold THoff (time t₂), the signal input to the frequency calculator 20 is switched from on to off. The on-threshold THon is greater than the off-threshold THoff.

When the variable band-pass filter 10 is not outputting the significant first signal Sig1, and the first signal Sig1 is similar to white noise, turning off the signal input to the frequency calculator 20 can prevent the phase-locked loop 21 (FIG. 3) of the frequency calculator 20 from tracking an incorrect frequency signal. By initializing the frequency calculator 20 in step SB6, it becomes possible to calculate the frequency from the initial state when the significant first signal Sig1 is subsequently input.

If the phase-locked loop 21 has characteristics that prevent malfunction even when the first signal Sig1 is similar to white noise, the input controller 50 may be omitted. The input controller 50 may also be provided with functions to adjust the gain of the input signal to the frequency calculator 20 and to adjust the sampling rate.

In the analysis in step SB1, the output signal of the variable band-pass filter 10 may be Fourier transformed, and in the determination of step SB2, if a peak is present in the frequency band of the target order n, it may be determined that the significant first signal Sig1 is being output.

Next, the excellent effects of the second example embodiment will be explained.

In the second example embodiment, by analyzing the signal input from the sensor 70 (FIG. 11), the order of the harmonic wave in a frequency band less susceptible to noise is determined as the target order n. Therefore, the frequency calculator 20 can calculate the frequency using the fundamental wave or a harmonic wave of the biological signal SigB that is less susceptible to noise among the fundamental wave and multiple harmonic waves. By determining the preferred target order n according to the noise conditions, the calculation accuracy of the frequency calculator 20 can be improved.

### Third Example Embodiment

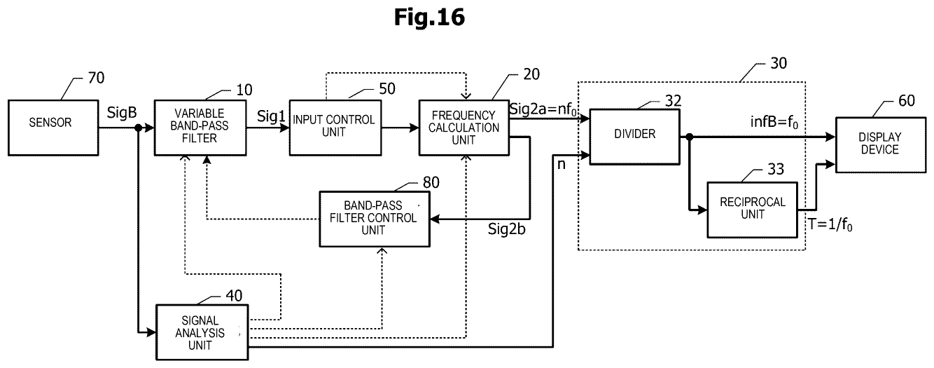

Next, a biological information measurement device according to a third example embodiment of the present invention will be explained with reference to the drawings from FIGS. 16 to 18. Hereinafter, the explanation for the components that are common to the biological information measurement device according to the second example embodiment, as explained with reference to the drawings from FIGS. 11 to 15, will be omitted.

FIG. 16 is a block diagram of the biological information measurement device according to the third example embodiment. In the second example embodiment (FIG. 11), the second signal Sig2 output from the frequency calculator 20 and input to the biological information acquirer 30 is also input to the band-pass filter controller 80. In contrast, in the third example embodiment, the frequency calculator 20 outputs two signals, a calculation signal Sig2a and a control signal Sig2b, as the second signal Sig2.

The calculation signal Sig2a is input to the biological information acquirer 30, while the control signal Sig2b is input to the band-pass filter controller 80. The values of the calculation signal Sig2a and the control signal Sig2b both track and fluctuate with variations in the frequency of the tracking signal Sigt, but the pattern of fluctuation differs between the two.

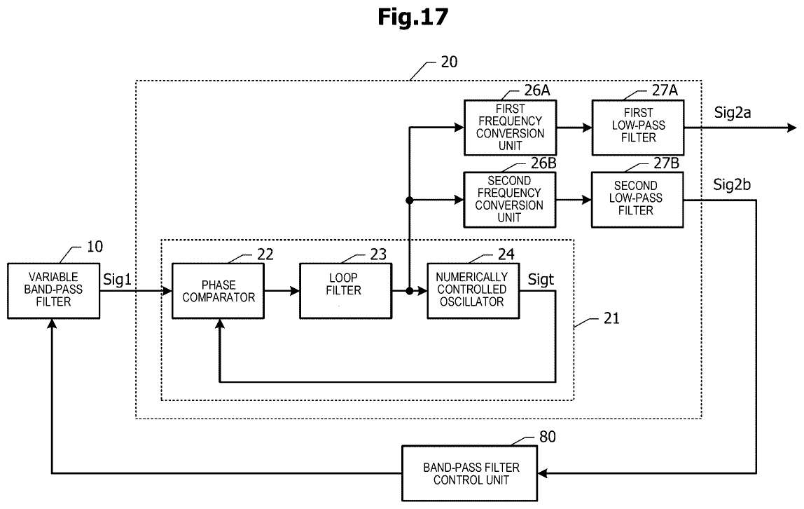

FIG. 17 is a block diagram of the frequency calculator 20 of the biological information measurement device according to the third example embodiment. The frequency calculator 20 of the biological information measurement device according to the second example embodiment (FIG. 11) includes one frequency converter 26 and one low-pass filter 27, as illustrated in FIG. 3. In contrast, the frequency calculator 20 of the biological information measurement device according to the third example embodiment includes a first frequency converter 26A, a second frequency converter 26B, a first low-pass filter 27A, and a second low-pass filter 27B.

The control value of a control signal output from the loop filter 23 and input to the numerically controlled oscillator 24 is input to the first frequency converter 26A and the second frequency converter 26B. The first frequency converter 26A and the second frequency converter 26B each convert the control value of the control signal to frequency information of the tracking signal Sigt of the current phase-locked loop 21. These items of frequency information converted by the first frequency converter 26A and the second frequency converter 26B are respectively input to the first low-pass filter 27A and the second low-pass filter 27B. Depending on the configuration of the loop filter 23 and the numerically controlled oscillator 24, the output of the loop filter 23 may include frequency information. In such a case, the first frequency converter 26A and the second frequency converter 26B are unnecessary.

The first low-pass filter 27A and the second low-pass filter 27B filter the input items of frequency information to output the calculation signal Sig2a and the control signal Sig2b, respectively.

For example, the frequency range of the numerically controlled oscillator 24 is greater than or equal to about 1 Hz and less than or equal to about 7 Hz. For example, a fourth-order IIR-type filter is used as the first low-pass filter 27A, and its cutoff frequency is about 1.0 Hz. For example, a fourth-order IIR-type filter is used as the second low-pass filter 27B, and its cutoff frequency is about 0.5 Hz. As described above, the cutoff frequency of the first low-pass filter 27A and the cutoff frequency of the second low-pass filter 27B are different from each other.

If the output delays of the first low-pass filter 27A and the second low-pass filter 27B are not a problem or if steep cutoff characteristics are not required, FIR filters may be used as the first low-pass filter 27A and the second low-pass filter 27B.

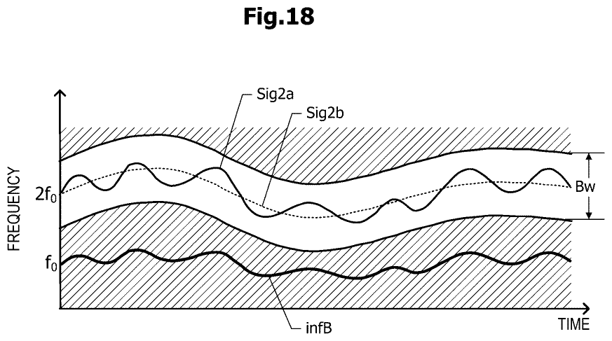

FIG. 18 is a graph illustrating an example of the temporal variation of the calculation signal Sig2a and the control signal Sig2b included in the second signal Sig2, the biological information infB, and the passband Bw of the variable band-pass filter 10. The horizontal axis represents time, and the vertical axis represents frequency. In FIG. 18, regions outside the range of the passband Bw are marked with hatching. FIG. 18 illustrates, for example, a case where the target order n of the harmonic wave that the frequency calculator 20 tracks and calculates is 2.

Since the cutoff frequency of the second low-pass filter 27B is lower than the cutoff frequency of the first low-pass filter 27A, the value of the control signal Sig2b fluctuates more gradually than the value of the calculation signal Sig2a. Since the center frequency of the passband Bw of the variable band-pass filter 10 tracks variations in the control signal Sig2b and varies accordingly, the center frequency of the passband Bw also varies gradually.

If the amplitude of the radio frequency components of the frequency nf₀ of the tracking signal Sigt (especially the frequency components higher than the frequency of the control signal Sig2b) is smaller than the bandwidth of the passband Bw, even a gradual variation in the center frequency Bw will not cause the value of the calculation signal Sig2a to fall outside the passband BW. Alternatively, if noise superimposed on the biological signal SigB (FIG. 16) is not large and it is possible to broaden the bandwidth of the passband Bw of the variable band-pass filter 10, even if the center frequency of the passband Bw is varied gradually, the value of the calculation signal Sig2a is unlikely to fall outside the passband Bw. The biological information infB, that is, the heartbeat frequency, can be obtained from the value of the calculation signal Sig2a which falls within the passband Bw.

Next, the excellent effects of the third example embodiment will be explained.

In the third example embodiment, the cutoff frequency of the first low-pass filter 27A to generate the calculation signal Sig2a and the cutoff frequency of the second low-pass filter 27B to generate the control signal Sig2b can be set independently of each other. Since it is only necessary to design the bandwidth of the first low-pass filter 27A and the second low-pass filter 27B based on the tracking characteristics required for the calculation signal Sig2a and the tracking characteristics required for the control signal Sig2b, the bandwidth design of the low-pass filters becomes easier, compared to a configuration where the two are processed with a single low-pass filter.

As explained with reference to FIG. 18, it is possible to make the variation in the value of the control signal Sig2b more gradual than the variation in the value of the calculation signal Sig2a. If the amplitude of the radio frequency components of the frequency nf₀ of the tracking signal Sigt is smaller than the bandwidth of the passband Bw, even if the center frequency of the passband Bw is varied gradually, the value of the calculation signal Sig2a will not fall outside the passband Bw. Therefore, without needing to make fine adjustments to the center frequency of the passband Bw of the variable band-pass filter 10, it is possible to capture the subtle fluctuations in the frequency 2f₀ of the tracking signal Sigt. This makes it possible to measure the subtle fluctuations in heartbeat frequency.

Additionally, by gradually varying the center frequency of the passband Bw of the variable band-pass filter 10, phase changes and the like caused by varying the center frequency of the passband Bw are less likely to occur. Therefore, the degradation in the frequency accuracy of the tracking signal Sigt caused by phase changes and the like is reduced or prevented. As a result, it becomes possible to measure the biological information infB with high accuracy.

Next, a modification of the third example embodiment will be explained.

In the third example embodiment, the cutoff frequency of the second low-pass filter 27B to generate the control signal Sig2b is set lower than the cutoff frequency of the first low-pass filter 27A to generate the calculation signal Sig2a. Conversely, it is acceptable to set the cutoff frequency of the first low-pass filter 27A to generate the calculation signal Sig2a lower than the cutoff frequency of the second low-pass filter 27B to generate the control signal Sig2b.

For example, while it is necessary to quickly change the center frequency of the passband Bw of the variable band-pass filter 10 in response to fluctuations in heartbeat frequency, if rapid changes are not required or desired for the heartbeat frequency measurements, it is preferable to set the cutoff frequency of the first low-pass filter 27A lower than the cutoff frequency of the second low-pass filter 27B.

As in the third example embodiment or its modification, the cutoff frequencies of the first low-pass filter 27A and the second low-pass filter 27B may be determined according to the required specifications and circumstances.

The above-described example embodiments are merely examples, and partial substitutions or combinations of the configurations illustrated in different example embodiments are possible. The same or similar advantageous operational effects from the same or similar configurations across multiple example embodiments will not be described sequentially for each example embodiment. Furthermore, the present invention is not limited to the example embodiments described above. For example, various modifications, improvements, combinations, and so on will be obvious to those skilled in the art.

While example embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A biological information measurement device comprising:
a variable band-pass filter to receive a biological signal with a harmonic structure;
a frequency calculator to receive a first signal that has passed through the variable band-pass filter and output a second signal including information related to a frequency of the received first signal;
a biological information acquirer to acquire biological information from the second signal; and
a band-pass filter controller configured or programmed to shift a passband of the variable band-pass filter based on the information related to the frequency included in the second signal.

2. The biological information measurement device according to claim 1, wherein
the frequency calculator includes a phase-locked loop to generate a tracking signal synchronized with the input first signal; and
the band-pass filter controller is configured or programmed to shift the passband of the variable band-pass filter in response to variations in frequency of the tracking signal.

3. The biological information measurement device according to claim 1, further comprising:
a signal analyzer to, based on the biological signal received by the variable band-pass filter, select one of a fundamental wave and a plurality of harmonic waves included in the biological signal as a target signal, and set the passband of the variable band-pass filter so as to pass a signal in a frequency band including a frequency of the target signal; wherein
the signal analyzer is configured to:
provide the frequency calculator with information used as a basis to output the second signal including information related to a frequency of the first signal; and
provide the biological information acquirer with information used as a basis to acquire the biological information from the second signal.

4. The biological information measurement device according to claim 3, wherein the biological information acquirer is configured to acquire the biological information by obtaining a fundamental frequency of the biological signal input to the variable band-pass filter based on the information related to the frequency of the first signal included in the second signal and an order of the target signal.

5. The biological information measurement device according to claim 2, wherein
the frequency calculator is configured to output a calculation signal and a control signal as the second signal;
the biological information acquirer is configured to acquire the biological information from the calculation signal;
the band-pass filter controller is configured or programmed to shift the passband of the variable band-pass filter based on information related to a frequency included in the control signal; and
the frequency calculator further includes a first low-pass filter and a second low-pass filter to receive frequency information of the tracking signal output from the phase-locked loop and output the calculation signal and the control signal, respectively.

6. The biological information measurement device according to claim 1, further comprising an acceleration sensor to obtain a ballistocardiogram.

7. The biological information measurement device according to claim 1, further comprising a display to display information acquired by the biological information acquirer.

8. The biological information measurement device according to claim 1, further comprising a sensor to detect the biological signal.

9. The biological information measurement device according to claim 8, wherein the biological signal includes a heartbeat signal.

10. The biological information measurement device according to claim 2, wherein the frequency calculator includes a frequency converter and a low-pass filter.

11. The biological information measurement device according to claim 2, wherein the phase-locked loop includes a phase comparator, a loop filter, and a numerically controlled oscillator.

12. The biological information measurement device according to claim 1, wherein the frequency calculator includes a multiplier phase-locked loop.

13. A variable filter circuit comprising:
a variable band-pass filter with a variable passband;
a phase-locked loop to generate a tracking signal synchronized with a phase of a signal that has passed through the variable band-pass filter; and
a band-pass filter controller configured or programmed to vary a passband of the variable band-pass filter based on a frequency of the tracking signal.
